(19) United States
(12) Patent Application Publication
(10) Pub. No.: US 2002/0005196 A1
(43) Pub. Date: Jan. 17, 2002

(54) Portable inhalator compressor device

(75) Inventor: Stephan Brugger, Starnberg (DE)

Correspondence Address: MERCHANT & GOULD PC, P.O. BOX 2903, MINNEAPOLIS, MN, 55402-0903, US

(73) Assignee: PARI GmbH Spezialisten fur effektive Inhalation, Starnberg (DE)

(21) Appl. No.: 09/875,377

(22) Filed: Jun. 6, 2001

Related U.S. Application Data
(63) Continuation of application No. 08/947,595, filed on Oct. 9, 1997, now abandoned.

(30) Foreign Application Priority Data
Nov. 15, 1994 (DE) G 94 18 334.1

(51) Int. Cl. A61M 11/00; B05B 17/06; A61M 15/00; A61M 16/10; F16K 11/00; G05D 11/02; A61M 16/00

(52) U.S. Cl. 128/200.16; 128/203.12; 128/200.21; 128/203.25

(57) ABSTRACT

The invention is directed to a portable inhalation device. This device is compact and portable and contains a compressor, an air filter and a power supply, and permits, together with an aerosol nebuliser, the mobile administration of drugs by inhalation.

### PORTABLE INHALATOR COMPRESSOR DEVICE

The subject matter of the invention is a portable inhalator compressor device, i.e. a compact, portable device which contains a compressor, an air filter and a power supply, and which permits together with an aerosol nebuliser the mobile administration of drugs by inhalation.

The administration of drugs in the form of an aerosol to be inhaled is a widespread form of therapy. In this respect it is a case of preparing an active ingredient in such a manner that a suspension of droplets or particles in air occurs, whereby the average diameter of these droplets or particles should be specifically adjustable, since the size determines where the active ingredient is primarily deposited in the body upon inhalation; larger droplets are mainly deposited in the mouth or pharyngeal cavity, whereas smaller particles reach into the bronchi and lungs. The droplets can occur directly from the fluid active ingredient solution to be administered, or also from solid particles of active ingredient suspended in fluid. Finally, it is also possible to mix a pulverous solid substance directly with air.

A known principle of the aerosol generation is the supply of air compressed by a compressor into a dispersion device, for example a nozzle, in which the air which is under a predetermined pressure is mixed with the fluid containing the active ingredient.

However, the known compressor inhalation devices have the disadvantage that the compressor is voluminous and determines the dimension of the complete device. Such devices are at least table devices or devices portable in a case, which although transportable are too large to be constantly carried around or used at arbitrary places.

However, in the course of many therapies, it is necessary that a patient inhales at short intervals, i.e. a number of times per day. This can be due to the fact that a constant administration of a drug is necessary to control symptoms, or also because a certain total dose for the day cannot be administered at one time. In this case, it is desirable that the patient can carry around an inhalation device so that his mobility is not restricted and he can follow his customary life rhythm.

For this purpose, pocket inhaltors have been available for a long time with hand pumps or pressure cartridges. However, these only permit a very rough dosage, and have only a slight degree of effectiveness with respect to the ratio of the applied amount of drug to the utilisable amount, so that such inhalators are not suitable for an exact dosage therapy. A compact, electrically operated device comprising an ultrasonic nebuliser is also known. However, such ultrasonic nebulisers are disadvantageous as compared to compressor-operated inhalation devices with respect to the scope of use with different drug formulations and the controllability of the aerosol application.

It is the object of the present invention to provide a compressor device for a portable, compressor-operated inhalation device which can be carried around by the patient comfortably and free of any problem, and which can be used at any time. The housing of the compressor device should be adapted in shape and size to the nebuliser receptacle.

This object is solved with a device according to the wording of the first claim.

The invention will now be explained on the basis of embodiments and with reference to the accompanying figures, the drawings showing the following:

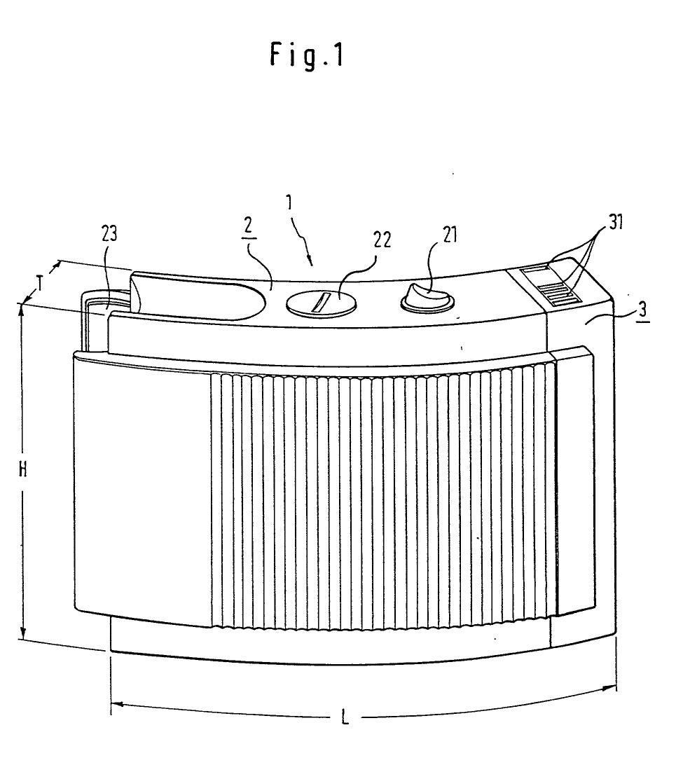

FIG. 1 shows a perspective view of the inhalator compressor device according to the invention;

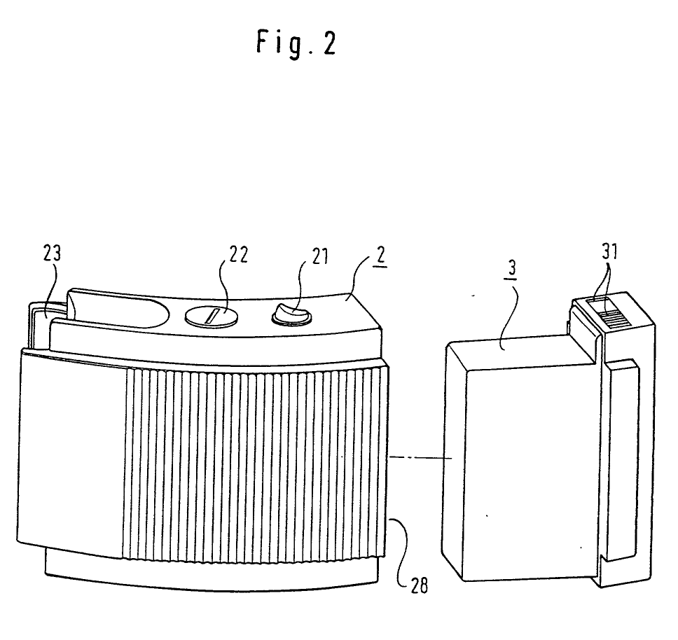

FIG. 2 shows the compressor housing and the removable power supply member;

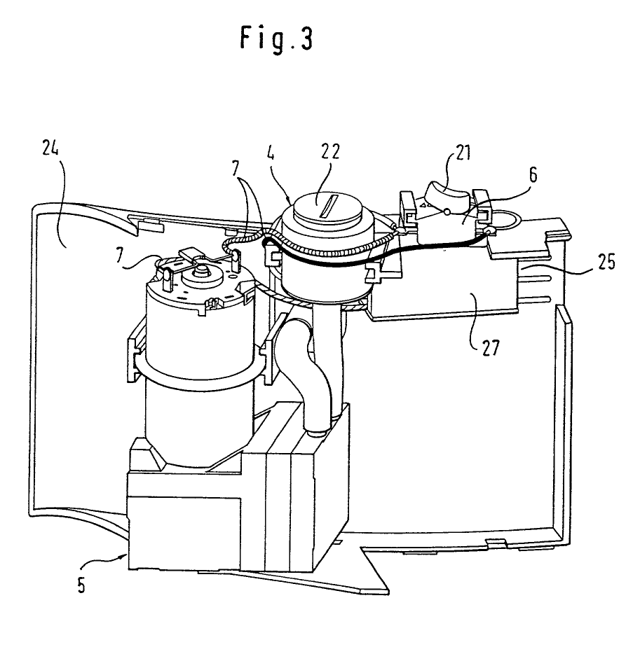

FIG. 3 shows the arrangement of the compressor, the air filter and the electric switch on the rear of the housing;

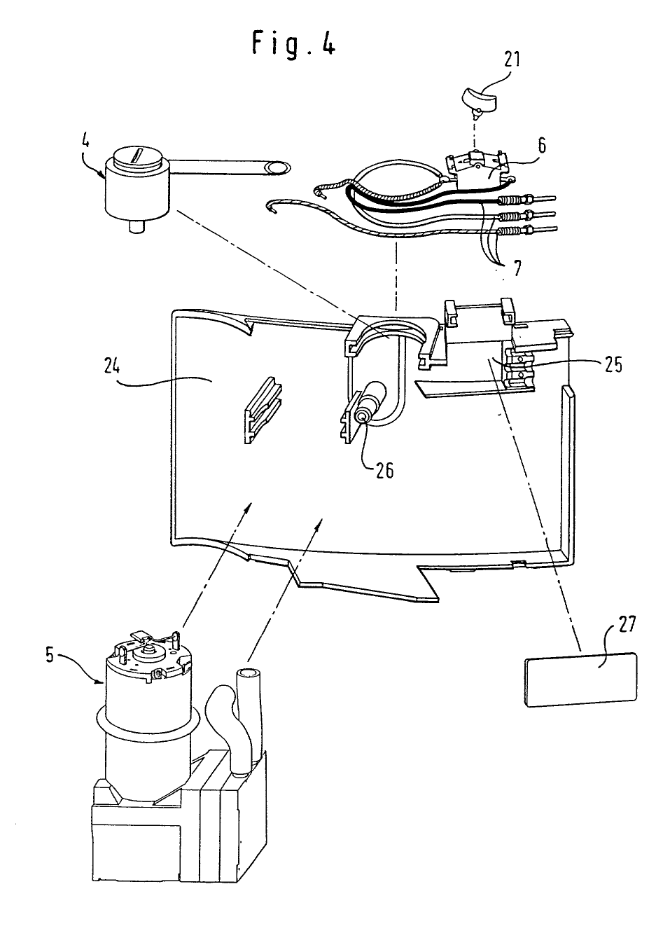

FIG. 4 shows the assembly of the members which lead to the arrangement of FIG. 3;

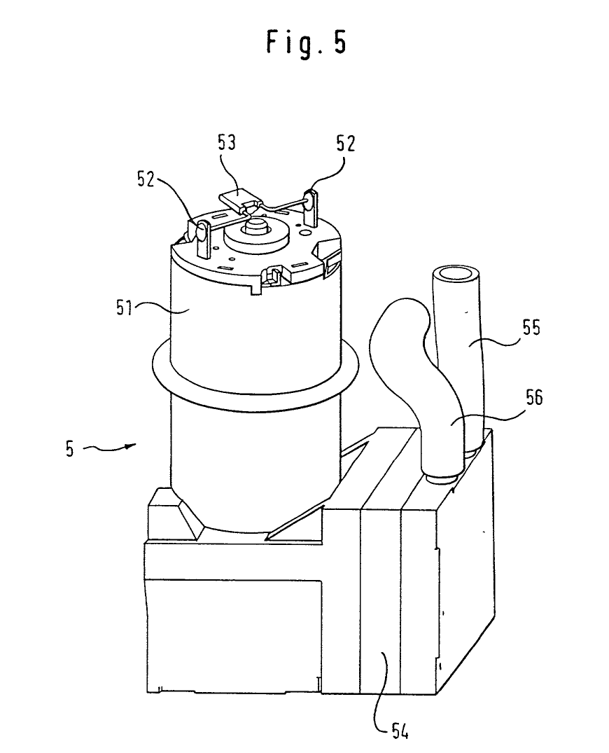

FIG. 5 shows the construction of the compressor;

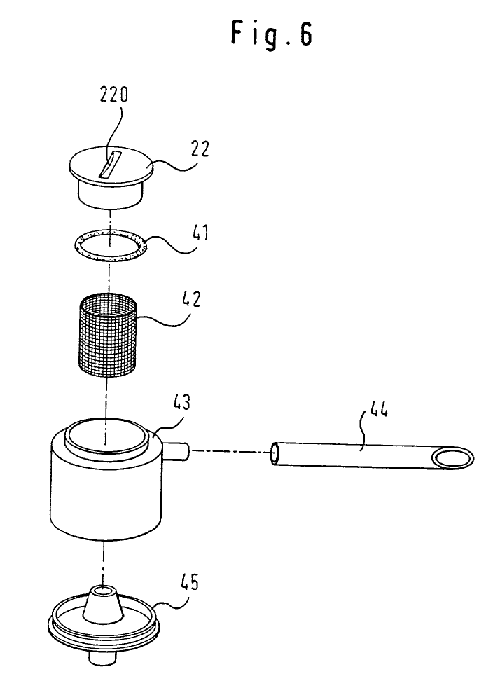

FIG. 6 shows the construction of the intake air filter.

In FIG. 1 a perspective view of the compressor device 1 is represented. It consists of a the compressor housing 2 and the power supply member 3. The compressor housing comprises a key switch 21 and an air filter receiving means 22.

On the end of the housing opposite the power supply member 3 a holding means 23 is provided for receiving a nebuliser receptacle (not illustrated). This permits the operation of the compressor together with diverse nebuliser receptacles and thus provides the invention with a high degree of flexibility with respect to the individual use for certain patients or specific therapies.

FIG. 2 shows that the power supply member 3 can preferably be removed from a recess 28 in the compressor housing 2. This power supply member 3 is generally a simple accumulator, which can be charged by an external charging device and provides information on its state of operation and load viadisplay members 31. However, the power supply member 3 can also be a mains adapter to connect the compressor device 1 to a main supply if available. This does not only include the public power supply mains (“socket”), but every possible kind of external power source, such as e.g. cigarette lighters in motor vehicles. Finally, a combined power supply member 2 is also conceivable in which accumulator and adapter are united in a single plug element. In this respect, the power supply member could be constructed in such a manner that upon operation of the device with a power supply from an external mains, the accumulator is simultaneously charged.

With this flexible power supply, it is ensured that the compressor device 1 is always ready for use, and simultaneously can always be operated with the external source of power momentarily available, sparing the accumulator.

In FIGS. 3 and 4, the internal construction of the compressor housing 2 is shown. An intake air filter 4, a compressor 5, an electric switch 6, and electric connection members 7, which are all arranged together on the rear segment 24 of the compressor housing 2 can be seen. On a power receiving member 25 closed with a plate 27 an electric connection to the power supply member 3 is provided, so that the electric connection members 7 connect the compressor 5 with the power supply member 3 via the switch 6 when the power supply member 3 is applied. In the simplest case, the switch 6 is a simple on/off switch, but a multi-stage switch can also be provided for multi-stage compressor operation.

In FIG. 5, the compressor 5 is shown. This includes an electromotor 51, which is supplied with power via terminals 52, these terminals 52 preferably being connected via a capacitor 53. Furthermore, the compressor 5 includes an actual compressor member 54, which contains the compression mechanism, together with an intake air tube or pipe 55 and an outlet tube or pipe 56. The intake air tube 55 is connected with the intake air filter 4, whereas the outlet tube 56 is connected with the output piece 26. The outlet piece 26 can be provided at its end facing away from the outlet tube 56 with an interface (not shown), via which a nebuliser can be connected to the compressor, but the interface can also be provided at the connection to the nebuliser (not shown) so that the end facing away from the outlet tube 56 is a simple opening.

FIG. 6 shows the construction of the intake air filter 4. It includes the filter receiving means 22, an 0-ring 41, the actual filter member 42, the filter housing 43, a suction tube 44 and the filter floor 45. These are assembled as indicated in FIG. 6, and connected with the compressor 5 and the rear segment 24 as shown in FIGS. 3 and 4. The filter receiving means 22 includes a turn lock fastener for fixing the filter member 42 in the filter housing 43. A coin for example fits into the notch 220 in the cover of the filter receiving means 22, so that every user of the device can change the filter element 42 when needed by untwisting the filter receiving means 22.

In the simplest case, the filter member 42 is a simple dust filter to avoid that the compressor blocks up, but it is also conceivable to use more specific filters which additionally preferably filter certain substances such as e.g. humidity. It is also possible to provide a sensor (not shown) to determine the state of charging, whereby a necessary change of the filter element 42 can then be indicated via a suitable display instrument, e.g. a control lamp.

The device is preferably to be of such a dimensioning that depth (T in FIG. 1) and height (H in FIG. 1) of the compressor housing 2 and of the power supply member 3 correspond with the depth and height of the nebuliser receptacle (not shown), so that a compact and easy to handle inhalation device results. This is achieved in the embodiment shown in the drawings in that the compressor 5 and the intake air filter 4 are superimposed in the center region of the housing, so that on the one side space remains for the nebuliser holding means 24 and on the other for the power supply member 3.

In a further preferred embodiment, the form of the compressor housing is slightly arcuate in its longitudinal stretch (L in FIG. 1), as indicated in FIG. 1. This simplifies the transport of the device on the body, e.g. on the hip, with the aid of a belt-bag or belt-holder. The housing is preferably provided with a ribbed surface to ensure a safe handling of the device, i.e. so that it does not easily slide out of the hand.

1. A portable inhalator compressor device (1), comprising:
a compressor housing (2) in which an electrically driven compressor (5) and an intake air filter (4) are arranged for filtering the air fed to the compressor (5), and which is equipped with a holding means (23) for receiving a nebuliser receptacle, the width of which essentially determines the width of the compressor housing, and
a power supply member (3), which can be introduced into a recess (28) in the compressor housing to supply the compressor with electric power.

2. A portable inhalator compressor device according to claim 1, characterized in that the power supply member (3) includes an accumulator.

3. A portable inhalator compressor device according to claim 1 or 2, characterized in that the power supply member (3) includes a mains adapter to supply the device with power from an external mains.

4. A portable inhalator compressor device according to one of the claims 1 to 3, characterized in that the shape of the compressor housing (2) and the power supply member (3) are selected such that the shape of the inhalator compressor device (1) is arcuate in its longitudinal stretch (L).

5. A portable inhalator compressor device according to one of the claims 1 to 4, characterized in that the holding means (23) is arranged on a narrow-sided end of the compressor housing (2) and the recess (28) is arranged on the opposite, narrow-sided end of said compressor housing (2).

6. A portable inhalator compressor device according to claim 5, characterized in that the length of the nebuliser housing substantially determines the height of the compressor housing (2).
